(19) United States
(12) Patent Application Publication
(10) Pub. No.: US 2024/0082116 A1
(43) Pub. Date: Mar. 14, 2024

(54) Self-Warming Hygienic Tissue

(71) Applicant: Jannette Bluford, San Francisco, CA (US)

(72) Inventor: Jannette Bluford, San Francisco, CA (US)

(21) Appl. No.: 17/943,998

(22) Filed: Sep. 13, 2022

(51) Int. Cl. A61K 8/02; A61K 8/11; A61Q 19/10

(52) U.S. Cl. CPC A61K 8/0208; A61K 8/11; A61Q 19/10; A61K 2800/242

(57) ABSTRACT

A self-warming hygienic tissue system may be provided utilizing a hygienic tissue having a first and second surface. The first surface may have a first plurality of ridges and a first plurality of troughs such that each of the first plurality of ridges may be disposed between a pair of troughs of the first plurality of troughs and each of the first plurality of troughs may be disposed between a pair of ridges of the first plurality of ridges. Further, the second surface may have a second plurality of ridges and a second plurality of troughs such that each of the second plurality of ridges are disposed between a pair of troughs of the second plurality of troughs and each of the second plurality of troughs are disposed between a pair of ridges of the second plurality of ridges.

### BACKGROUND OF THE INVENTION

The multi-billion dollar hygienic tissue market has seen significant growth in the wake of ongoing global health crises which have made the general public more aware of public health best practices. In use, hygienic tissues are designed to decrease the transmissibility of pathogenic agents while also providing the user with a sense of cleanliness in the circumstances of meals, public interaction and the like. Hygienic tissues are also used to remove gross filth, dirt, debris and the like from the surface of the user's skin in the circumstances, for example, of cleaning up a child's dirty diaper and the like.

When applying a hygienic tissue directly to a user's skin surface for removal of filth, dirt, debris and the like, it is ideal that the hygienic tissue removes as much of the contaminating material as possible in the first pass of the wipe across the skin surface. Often, the structure of the hygienic tissue substrate determines the ability for the hygienic tissue to optimally remove the contaminating material from the user's skin surface. For example, a comparatively flat substrate structure is less effective at removing contaminants from a user's skin surface relative a non-flat substrate structure. It would be advantageous to provide a hygienic tissue that comprises a non-flat substrate structure in order to optimally remove contaminants from the user's skin surface.

During application of a hygienic tissue directly to a user's skin surface, the tissue substate often comprises a wet carrier solution that aides in removal of contaminants from the user's skin surface. However, the wet carrier solution often creates a cold-to-the-touch feeling for the user when the applying the tissue substrate to the skin surface. This feeling leads to discomfort for the user when using the hygienic tissue and acts as a deterrent to use. If the wet carrier solution were to comprise a neutral temperature feeling when applied to the user's skin, then there would exist no discomfort during use. A neutral feeling may be achieved when the fluid being applied is at or near the temperature of the user's skin surface, e.g. roughly between 92 and 98 degrees. It would be advantageous to provide a hygienic tissue having a wet carrier solution that is at or near the user's skin surface temperature when applied to the skin surface to minimize user discomfort.

Given that it is advantageous to provide a hygienic tissue at or near a skin surface temperature, it is important to maintain temperature stability of the wet carrier solution before and during use of the hygienic tissue. The wet carrier solution is not able to maintain a temperature at or near the skin surface for a prolonged period of time given that the ambient environment is typically significantly below that of the skin surface. Therefore, the temperature should be held stable at or near the ambient environmental temperature prior to application to the skin surface, but should be raised at or near the skin surface temperature at the time of application to the skin surface. It would be advantageous to provide a hygienic tissue having a wet carrier solution that can have its contact temperature raised to at or near the user's skin surface temperature at a time selected by the user.

In order to provide a hygienic tissue that raises its contact temperature at a time selected by the user, a user-friendly means of activating the temperature of the wet carrier solution is required. Utilizing a heater or other external device would be inconvenient for use with the hygienic tissue given the portable nature of desired use of hygienic tissues. Therefore, a portable means of easily activating the temperature increase of the hygienic tissue by all users is required. It would be advantageous to provide a hygienic tissue that is temperature activated by physical abrasion of the tissue substate either by the fingers of the user grasping the tissue and/or by the dispensing mechanism housing the tissues.

### BRIEF DESCRIPTION OF THE DRAWINGS

FIG. 1 illustrates a perspective view of a tissue dispensing system comprising a self-warming hygienic tissue partially disposed within a tissue dispensing container in accordance with some embodiments of the present invention.

FIG. 2 illustrates an overview of a self-warming hygienic tissue being agitated between a user's fingers and thumb in accordance with some embodiments of the present invention.

FIG. 3 illustrates a detailed cross-sectional view of a portion of a self-warming tissue having serially-arranged interior pockets in accordance with some embodiments of the present invention.

FIG. 4 illustrates an overview of a process of releasing fluids from a microcapsule via agitation in accordance with some embodiments of the present invention.

FIG. 5 illustrates an overview of a process of forming a microcapsule via an encapsulation technique in accordance with some embodiments of the present invention.

### DETAILED DESCRIPTION OF THE INVENTION

Before describing the present invention in detail, it is to be understood that the invention is not limited to any one of the particular embodiments, which of course may vary. It is also to be understood that the terminology used herein is for the purpose of describing particular embodiments only, and therefore is not necessarily intended to be limiting. As used in this specification and the appended claims, terms in the singular and the singular forms “a,” “an,” and “the” include plural referents unless the context clearly dictates otherwise. Thus, for example, reference to “a self-warming hygienic tissue” also includes a plurality of self-warming hygienic tissues and the like.

In some embodiments, a self-warming hygienic tissue system is provided comprising: a hygienic tissue comprising a first surface and a second surface, wherein: the first surface comprises a first plurality of ridges and a first plurality of troughs, wherein: each of the first plurality of ridges are disposed between a pair of troughs of the first plurality of troughs, and each of the first plurality of troughs are disposed between a pair of ridges of the first plurality of ridges, the second surface comprises a second plurality of ridges and a second plurality of troughs, wherein: each of the second plurality of ridges are disposed between a pair of troughs of the second plurality of troughs, and each of the second plurality of troughs are disposed between a pair of ridges of the second plurality of ridges; a plurality of interior pockets disposed between the first surface and the second surface; and a plurality of microcapsules comprising one or more self-warming solution ingredients.

In some embodiments, the first surface and the second surface are coupled together at the locations of each of the first plurality of troughs and each of the second plurality of troughs.

In some embodiments, each of the first plurality of troughs are coupled to one of the second plurality of troughs.

In some embodiments, the first and second plurality of ridges and the first and second plurality of troughs form a shape of a square wave, a sinusoidal wave, a triangular wave, a pulse wave, a sawtooth wave or any combination thereof.

In some embodiments, each of the plurality of interior pockets are disposed between a pair of ridges of the first and second plurality of ridges.

In some embodiments, the plurality of interior pockets are serially arranged in a first direction.

In some embodiments, each of the plurality of interior pockets are arranged parallel one another in a second direction orthogonal to the first direction.

In some embodiments, the hygienic tissue is fabricated from cotton, viscose, cellulosic fibers, polyester, polypropylene, polymer resin, wood pulp or any combination thereof.

In some embodiments, the hygienic tissue comprises an aqueous solution absorbed therein.

In some embodiments, the plurality of microcapsules are disposed within the plurality of interior pockets.

In some embodiments, each of the plurality of microcapsules comprise a diameter of between 1 micron and 500 microns.

In some embodiments, the one or more self-warming solution ingredients contained in each hygienic tissue comprise a total volume of between 3 milliliters and 12 milliliters.

In some embodiments, each of the plurality of microcapsules are structurally robust up to a threshold rupturing force.

In some embodiments, each of the plurality of microcapsules are structurally robust up to a threshold temperature.

In some embodiments, the one or more self-warming solution ingredients produce an exothermic chemical reaction when exposed to environmental air.

In some embodiments, the exothermic chemical reaction raises a temperature of the hygienic tissue.

In some embodiments, the system further comprises a tissue dispensing container housing the hygienic tissue.

In some embodiments, the tissue dispensing container comprises a plurality of tissue retention structures.

In some embodiments, a self-warming hygienic tissue system is provided comprising: a hygienic tissue comprising a first surface and a second surface, wherein: the first surface comprises a first plurality of ridges and a first plurality of troughs forming a first periodic shape, the second surface comprises a second plurality of ridges and a second plurality of troughs forming a second periodic shape, and the first and second periodic shapes each form a sinusoidal shape; a plurality of interior pockets disposed between the first surface and the second surface, wherein: the plurality of interior pockets are serially arranged, and each of the plurality of interior pockets are arranged parallel relative to one another; and a plurality of microcapsules comprising one or more self-warming solution ingredients, wherein the one or more self-warming solution ingredients produce an exothermic chemical reaction when exposed to environmental air.

In some embodiments, a self-warming hygienic tissue system is provided comprising: a hygienic tissue comprising a first surface and a second surface, wherein: the first surface comprises a first periodic shape, the second surface comprises a second periodic shape, and the first surface is coupled to the second surface; a plurality of interior pockets disposed between the first periodic shape and the second periodic shape, wherein: the plurality of interior pockets are serially arranged, each of the plurality of interior pockets are arranged parallel relative to one another, and each of the plurality of interior pockets are disposed between pairs of opposing peaks of the first and second periodic shapes; and a plurality of microcapsules comprising one or more self-warming solution ingredients, wherein: the plurality of microcapsules are disposed within the plurality of interior pockets, the one or more self-warming solution ingredients produce an exothermic chemical reaction when exposed to environmental air, and the exothermic chemical reaction raises a temperature of the hygienic tissue.

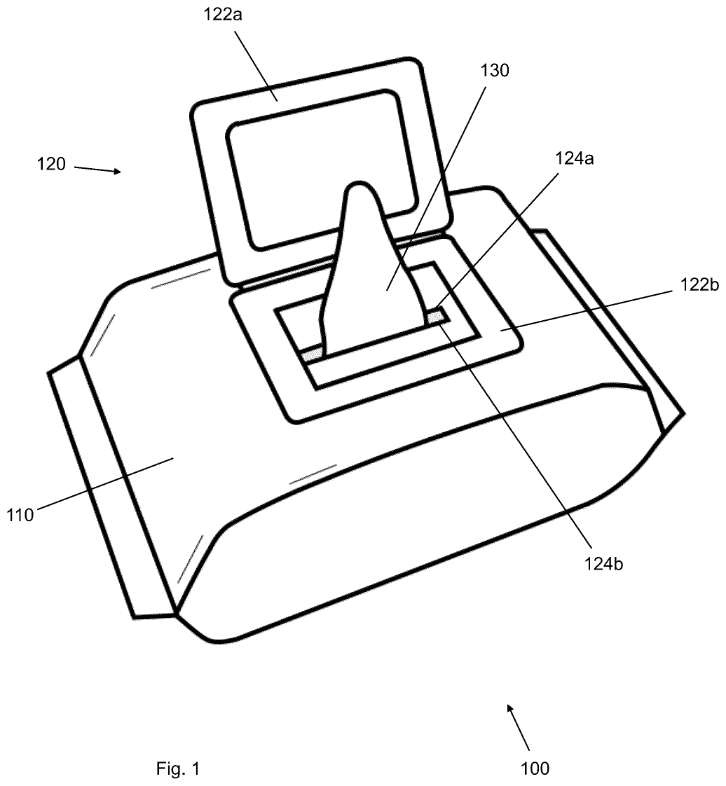

Exemplary embodiments of the present invention are illustrated in the accompanying figures. As shown in FIG. 1, a perspective view of a tissue dispensing system 100 comprising a self-warming hygienic tissue 130 partially disposed within a tissue dispensing container 110 is provided. The tissue dispensing system 100 may comprise the tissue dispensing container 110 having a top surface, a bottom surface and at least two side surfaces. A closure mechanism 120 may be disposed upon the top surface of the container 110 to allow a user access to the self-warming hygienic tissue 130. The closure mechanism 120 may comprise a top portion 122a and a bottom portion 122b.

The top portion 122a may be coupled to the bottom portion 122b via a hinge or like structure such that the top portion 122a may be rotated between an open position, as shown in FIG. 1, and a closed position. The closure mechanism 120 may further comprise a first retention structure 124a opposite a second retention structure 124b. Both of the first and second retention structures 124a, 124b may be contained within the bottom portion 122b such that the self-warming hygienic tissue 130 is retained in an upright position between the first and second retention structures 124a, 124b.

In use, a plurality of self-warming hygienic tissues 130 may be stored within the interior cavity of the tissue dispensing container 110. Upon dispensing of the given hygienic tissue 130 retained between the first and second retention structures 124a, 124b for use by a user, the tissues 130 stacked within the container 110 may be arranged in such a manner so as to automatically secure the next tissue 130 in the stack within the first and second retention structures 124a, 124b. Thereby, each dispensation of the retained tissue 130 may manipulate the next tissue 130 in the stack between the first and second retention structures 124a, 124b iteratively as desired by the user.

The self-warming hygienic tissue 130 may have a temperature change capability that may be triggered by mechanical and/or temperature agitation of the self-warming hygienic tissues 130. In one embodiment, mechanical agitation may take the form of frictional abrasion of the tissue 130 structure by the exterior surfaces of the first and second retention structures 124a, 124b resulting from dispensation of the tissue 130 through the aperture between the first and second retention structures 124a, 124b. Specifically, as the tissue 130 is pulled through the aperture, the exterior structural surfaces of the first and second retention structures 124a, 124b mechanically abrade the exterior surface of tissue 130 due to the force applied by the user in pulling the tissue 130 and the the coefficient of friction between the first and second retention structures 124a, 124b and the tissue 130 material.

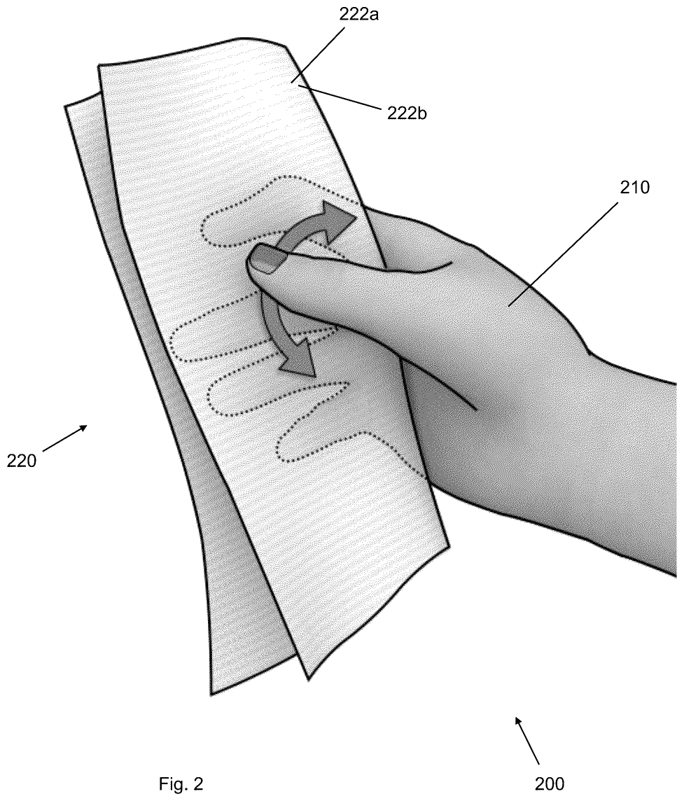

As shown in FIG. 2, an overview of an agitation process 200 of a self-warming hygienic tissue 220 via mechanical abrasion between a user's fingers and thumb is provided. The agitation process 200 may involve a user's hand 210 grasping the self-warming hygienic tissue 220 between the user's fingers and thumb. The user may then rub their fingers and thumb together in a reciprocating manner that causes mechanical abrasion between one or more portions of the hygienic tissue 220, the user's fingers and/or the user's thumb due to the force applied by the user's hand 210 and the coefficient of friction between one or more portions of the hygienic tissue 220, the user's fingers and the user's thumb.

In order to increase the coefficient of friction between one or more portions of the hygienic tissue 220, the user's fingers and the user's thumb, the tissue 220 may comprise a series of alternating parallel rows of ridges 222a and troughs 222b. Specifically, each ridge 222a may extend across the entire length dimension of the tissue 220 and each trough 222b may similarly extend across the entire length dimension of the tissue 220. Each ridge 222a may be followed by a trough 222b and may be disposed in manner parallel thereto, thereby forming a series of alternating parallel rows of ridges 222a and troughs 222b as shown in FIG. 2.

Generally, the agitation process 200 is designed to generate a rupture force exceeding a predetermined threshold that is sufficient to compromise the structural integrity of a plurality of microcapsules disposed within the interior portion of each of the parallel rows of ridges 222a. In some embodiments, the threshold rupturing force may be between 25 millinewtons and 300 millinewtons. A threshold rupturing force below 25 millinewtons would render the microcapsules too easily ruptured thereby rendering the tissue 220 non-functional. A threshold rupturing force above 300 millinewtons would render the microcapsules too difficult to rupture thereby rendering the tissue 220 non-functional. Upon rupturing of each microcapsule, a predetermined amount of thermal self-warming solution is released therefrom and exposed to environmental air at which point an exothermic chemical reaction takes place between the thermal self-warming solution and the environmental air.

While mechanical abrasion may be the means of agitating the self-warming hygienic tissue 220 and thereby rupturing the structural integrity of the microcapsules, increasing the temperature of the tissue 220 may be another means of agitation. For example, when the user grasps the self-warming hygienic tissue 220 in the user's hand 210, the tissue 220 may be heated beyond a threshold temperature by the natural body heat of the user's hand 210 and thereby be thermally agitated in the associated locations where tissue 220 temperature is increasing. By local application of external heat from the user's hand beyond the threshold temperature, the structure of the microcapsule may become compromised and thereby may release the thermal self-warming solution contained therein.

In some embodiments, the threshold temperature is between 85 degrees Fahrenheit and 95 degrees Fahrenheit. Such a range of threshold temperatures is advantageous as it does not rupture the microcapsules under typical residential temperatures which typically fall under 85 degrees Fahrenheit but also allows the user's body heat to trigger the rupturing of the microcapsule as many areas of the human body comprise a surface temperature above 85 degrees Fahrenheit but below 95 degrees Fahrenheit. Temperatures below 85 degrees Fahrenheit would too easily trigger structural rupturing of the microcapsules while temperatures above 95 degrees Fahrenheit would be too difficult to trigger the same.

In some embodiments, the user's hand 210 may apply both mechanical and thermal agitation to the tissue 220 to further increase the level of agitation to the tissue 220 which expedites the self-warming process of the tissue 220. For instance, local application of external heat from the user's hand may partially degrade the structural integrity of the microcapsules which thereby render the microcapsules more susceptible to structural rupturing upon mechanical agitation by the user's fingers and thumb which are already applying thermal energy by their grasping of the tissue 220.

The exothermic release of thermal energy from this chemical reaction provides the heat to enable the self-warming properties of the tissue 220. Upon the exothermic release of thermal energy, the surface temperature of the tissue 220 where microcapsules have been ruptured may be raised from room temperature to a skin temperature range. The skin temperature range may comprise temperatures between 85 degrees and 100 degrees Fahrenheit, but more preferably between 85 degrees and 95 degrees and most preferably between 85 degrees and 90 degrees. Tissue 220 temperatures below 85 degrees may feel cold to the touch of the user's skin which generates discomfort for the user during use. Tissue 220 temperatures above 100 degrees may feel hot to the touch of the user's skin which generates discomfort for the user during use.

Generally, temperatures between 85 degrees and 100 degrees allow for the tissue 220 temperature to be within the typical range of the user's surface skin temperature at various locations where the self-warming hygienic tissue 220 may be used. On average, human skin surface temperature rests at around 91 degrees Fahrenheit and so the preferred range of between 85 degrees and 95 degrees and the more preferred range of between 85 degrees and 90 degrees are more closely aligned with the user's skin surface temperature. Moreover, it is preferred to err on the underside of the average human skin surface temperature as it is preferred to prevent a potential burning sensation to the user's skin due to overheating of the tissue 220. Further, certain portions of the human body have a below average skin surface temperature and so utilizing a tissue temperature range slightly below the average human skin surface temperature, such as the range between 85 degrees and 90 degrees, would be advantageous.

The thermal self-warming solution may comprise any suitable ingredients that enable the self-warming properties of the tissue 220. In some embodiments, the thermal self-warming solution may comprise one or more ingredients including activated charcoal, vermiculite, iron powder, sodium chloride and the like or any combinations thereof. Each ingredient of the thermal self-warming solution may be included in equal parts within an error range of about 10%. An error range above 10% may alter the desired heating effect of the exothermic chemical reaction as the thermal self-warming solution reacts with environmental air. Specifically, excess error may cause the tissue 220 to heat to a temperature outside of the range of between 85 degrees and 100 degrees.

Each tissue 220 may comprise a total of between 3 milliliters and 12 milliliters of thermal self-warming solution collectively contained within the plurality of microcapsules disposed within the cavity of the parallel rows of ridges 222a. Preferably, each tissue 220 may comprise a total of between 5 milliliters and 10 milliliters of self-warming solution, but more preferably between 6 milliliters and 9 milliliters. Any total less than 3 milliliters does not provide enough moisture to the tissue 220 material in order to allow the tissue 220 to be comfortable to the touch of the skin of the user. Any total more than 12 milliliters provides too much moisture to the tissue 220 material such that use of the tissue 220 by the user becomes uncomfortable and messy to the touch of the skin of the user.

The fabrication material of the tissue 220 may comprise one or more of cotton, viscose, cellulosic fibers, polyester, polypropylene, polymer resin, wood pulp and the like or any combination thereof. Cotton is advantageous as a fabrication material as it provides enhanced liquid absorption and liquid retention properties while still being soft to the touch of the user's skin. Viscose is advantageous as a fabrication material as it provides enhanced liquid absorption and strength properties while being ideal for sensitive skin. Polyester fibers are advantageous as a fabrication material as they provide the relatively highest level of strength but are not as soft as other materials. Wood pulp is advantageous as a fabrication material as it provides a high surface area material on the micro scale while also having relatively high levels of cleaning ability.

The self-warming hygienic tissue 220 material may comprise an aqueous solution absorbed therein in order to make the tissue 220 wet to the touch of the user's skin. The aqueous solution, in part, may comprise propylene glycol, Aloe barbadensis, citric acid, glycerin and the like or any combination thereof. These ingredients are present in the material of the tissue 220 that defines the structure thereof but is not present within the microcapsules. Rather, the microcapsules maintain structural separation between the thermal self-warming solution and the aqueous solution until structural rupturing of the microcapsules.

While the tissue 220 is illustrated as having a series of alternating parallel rows of ridges 222a and troughs 222b, it is contemplated as a part of this disclosure that the tissue 220 may have a checkerboard pattern of ridges 222a and troughs 222b that are formed in a similar manner to that illustrated in FIG. 2. Specifically, the fabrication process by which the parallel rows of ridges 222a and troughs 222b are formed is the same or similar process that is used to form a set of parallel columns of ridges 222a and troughs 222b that are arranged orthogonal to that of the parallel rows of ridges 222a and troughs 222b, thereby forming a checkerboard pattern of ridges 222a and troughs 222b. The checkerboard pattern is advantageous in some embodiments to provide additional surface contour which increases the coefficient of friction of the tissue 220 with respect to a work surface or a user's skin surface which allows greater removal of debris and related contaminants and filth from those surfaces.

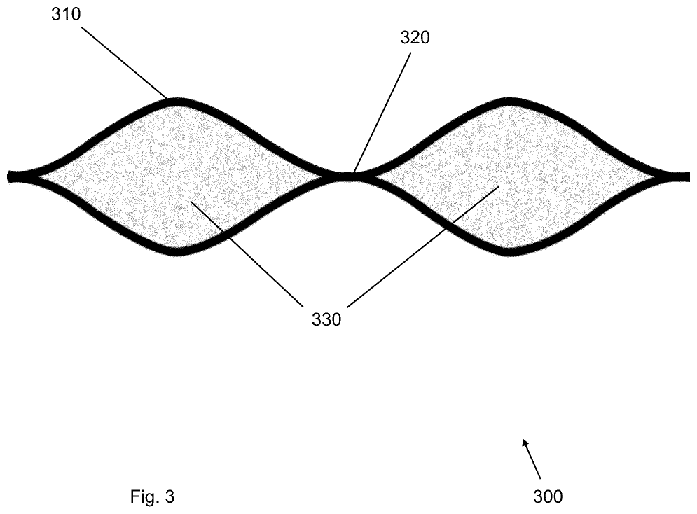

As shown in FIG. 3, a detailed cross-sectional view of a portion of a self-warming tissue 300 having serially-arranged interior pockets 330 is provided. The self-warming tissue 300 may comprise a plurality of parallel rows of ridges 310 separated by an associated plurality of parallel rows of troughs 320 such that parallel rows of ridges 310 and troughs 320 alternative in sequence along the length of the tissue 300. Generally, the alternating parallel rows of ridges 310 and troughs 320 form a modified sinusoidal shape that may comprise standard, rectangular and/or triangular sinusoidal shapes and the like or any combinations thereof.

Such a shape is advantageous to provide a cost-effective means of producing the tissue 300 while also providing a plurality of serially-arranged interior pockets 330 formed within each row of ridges 310 as shown in FIG. 3. Each of the serially-arranged interior pockets 330 may comprise a plurality of microcapsules disposed therein as illustrated in FIG. 3 where each microcapsule may comprise a predetermined amount of thermal self-warming solution. Each of the microcapsules may release the thermal self-warming solution contained therein via mechanical and/or temperature agitation as discussed with regard to FIG. 2.

The thickness of the tissue 300, i.e. the distance between opposing ridges 310, will typically depend upon the diameter size of the microcapsules and the number of microcapsules contained within each interior pocket 330. For instance, as the diameter of the microcapsules increases, the thickness of the tissue 300 must increase in order to prevent the tissue 300 from having a sandy feel to the touch of the user's skin which causes discomfort during use. The length and width of the tissue 300 may be arbitrary and dependent upon the use case for the user. In some embodiments, the tissue 300 may be structurally connected to successive tissues in the dispensing container by a perforation line which allows the user to decide how large of a tissue 300 they would like to use by choosing whether or not to separate the given tissue 300 from each successive tissue in the stack.

While the tissue 300 is illustrated as having a series of alternating parallel rows of ridges 310 and troughs 320, it is contemplated as a part of this disclosure that the tissue 300 may have a checkerboard pattern of ridges 310 and troughs 320 that are formed in a similar manner to that illustrated in FIG. 3. Specifically, the fabrication process by which the parallel rows of ridges 310 and troughs 320 are formed is the same or similar process that is used to form a set of parallel columns of ridges 310 and troughs 320 that are arranged orthogonal to that of the parallel rows of ridges 310 and troughs 320, thereby forming a checkerboard pattern of ridges 310 and troughs 320. The checkerboard pattern is advantageous in some embodiments to provide additional surface contour which increases the coefficient of friction of the tissue 300 with respect to a work surface or a user's skin surface which allows greater removal of debris and related contaminants and filth from those surfaces.

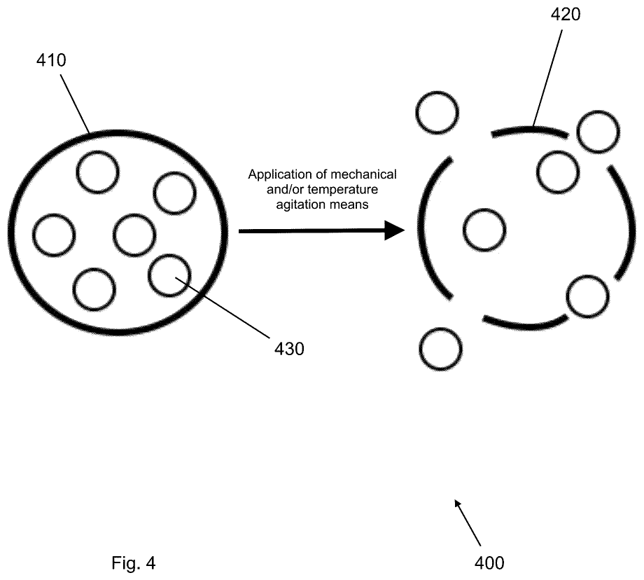

As shown in FIG. 4, an overview of a process 400 of releasing fluids 430 from a microcapsule 410 via agitation is provided. The process 400 may utilize application of mechanical and/or temperature agitation means in order to transform an in-tact microcapsule 410 into a structurally-compromised microcapsule 420. The structurally-compromised microcapsules 420 may allow the fluids 430 to escape therefrom, as illustrated in FIG. 4, and chemically interact with the given surrounding environmental air and tissue materials. Such means of mechanical and/or temperature agitation may include the frictional force generated during the dispensation of the tissue from a package, the frictional force generated during handling of the tissue by the user, the application of temperate via the body heat from the user's hand and the like or any combinations thereof.

In some embodiments, the fluids 430 may comprise one or more thermal self-warming solution ingredients as described with regard to FIG. 2. Consequently, the fluids 430 chemically interacting with environmental air may trigger an exothermic reaction that provides heat to the self-warming hygienic tissue. While the fluid 430 molecules are illustrated as being roughly identical, it is understood that the fluids 430 may comprise one or more self-warming solution ingredients including activated charcoal, vermiculite, iron powder, sodium chloride and the like or any combinations thereof.

Each microcapsule 410 may take the form of a microencapsulated fluid 430 as shown in FIG. 4. Further, the microcapsules 410 may comprise a particle size of between 1 micron and 500 microns. The fluids 430 may be encapsulated by the microcapsules 410 which may be fabricated from a natural or synthetic polymeric membrane. The encapsulation process may utilize a cross-linking polymerization reaction and may be employed in order to protect the fluids 430 from oxidation and evaporation.

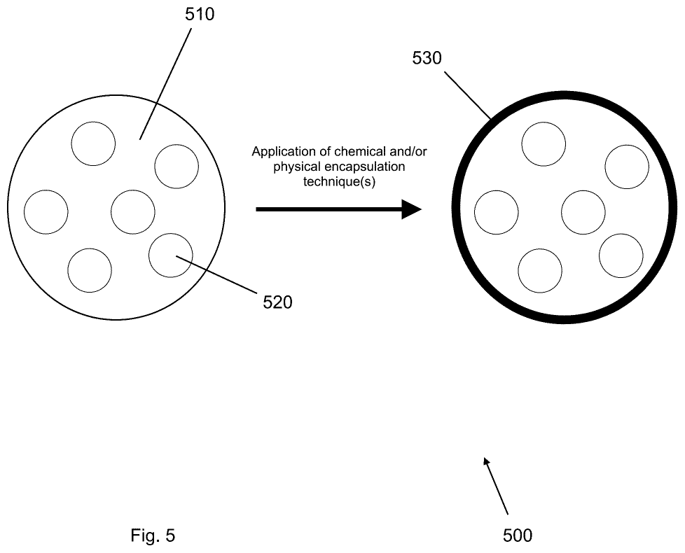

As shown in FIG. 5, an overview of a process 500 of forming a microcapsule 530 via an encapsulation technique is provided. The process 500 may comprise disposing one or more fluids 520 in a microcapsule precursor solution. After application of chemical and/or physical encapsulation techniques, the microcapsule precursor solutions may form the rigid microcapsule 530 while leaving the fluids 520 disposed therein. Such encapsulation techniques may include coacervation, interfacial polymerization, in situ polymerization and the like or any combination thereof. Preferably, a polymerization reaction between a cross-linked component and an activator compound may be utilized in an emulsion environment in order to form the microcapsules 530 around their associated fluids 520.

The specification and drawings are to be regarded in an illustrative rather than a restrictive sense. However, it will be evident that various modifications and changes may be made thereunto without departing from the broader spirit and scope of the invention as set forth in the claims. Other variations are within the spirit of the present disclosure. Thus, while the disclosed techniques are susceptible to various modifications and alternative constructions, certain illustrated embodiments thereof are shown in the drawings and have been described above in detail. It should be understood, however, that there is no intention to limit the invention to the specific form or forms disclosed, but on the contrary, the intention is to cover all modifications, alternative constructions, and equivalents falling within the spirit and scope of the invention, as defined in the appended claims.

Throughout this disclosure, the phrase ‘modularly coupled’ and similar terms and phrases are intended to convey that any element of a given class of elements may be coupled to another given element and vice versa with equal effect. For example, any extension cord of a plurality of extension cords may be modularly coupled to another extension cord and vice versa with equal effect. Further, throughout this disclosure, the phrase ‘removably coupled’ and similar terms and phrases are intended to convey that a given element may be iteratively coupled to and removed from another given element as desired. For example, a male plug of a first extension cord may be removably coupled to a female plug of a second extension cord as desired.

The use of the terms “a,” “an,” “the,” and similar referents in the context of describing the disclosed embodiments (especially in the context of the following claims) are to be construed to cover both the singular and the plural, unless otherwise indicated or clearly contradicted by context. The terms “comprising,” “having,” “including,” and “containing” are to be construed as open-ended terms (i.e., meaning “including, but not limited to,”) unless otherwise noted. The term “connected,” where unmodified and referring to physical connections, is to be construed as partly or wholly contained within, attached to, or joined together, even if there is something intervening. Recitation of ranges of values are merely intended to serve as a shorthand method of referring individually to each separate value falling within the range, unless otherwise indicated and each separate value is incorporated into the specification as if it were individually recited. The use of the term “set” (e.g., “a set of items”) or “subset” unless otherwise noted or contradicted by context, is to be construed as a nonempty collection comprising one or more members. Further, unless otherwise noted or contradicted by context, the term “subset” of a corresponding set does not necessarily denote a proper subset of the corresponding set, but the subset and the corresponding set may be equal.

Conjunctive language, such as phrases of the form “at least one of A, B, and C,” or “at least one of A, B and C,” is understood with the context as used in general to present that an item, term, etc., may be either A or B or C, or any nonempty subset of the set of A and B and C, unless specifically stated otherwise or otherwise clearly contradicted by context. For instance, in the illustrative example of a set having three members, the conjunctive phrases “at least one of A, B, and C” and “at least one of A, B and C” refer to any of the following sets: {A}, {B}, {C}, {A, B}, {A, C}, {B, C}, {A, B, C}. Thus, such conjunctive language is not generally intended to imply that certain embodiments require at least one of A, at least one of B and at least one of C each to be present. In addition, unless otherwise noted or contradicted by context, the term “plurality” indicates a state of being plural (e.g., “a plurality of items” indicates multiple items). The number of items in a plurality is at least two, but can be more when so indicated either explicitly or by context.

The use of any examples, or exemplary language (e.g., “such as”) provided, is intended merely to better illuminate embodiments of the invention and does not pose a limitation on the scope of the invention unless otherwise claimed. No language in the specification should be construed as indicating any non-claimed element as essential to the practice of the invention.

Embodiments of this disclosure are described, including the best mode known to the inventors for carrying out the invention. Variations of those embodiments may become apparent to those of ordinary skill in the art upon reading the foregoing description. The inventors expect skilled artisans to employ such variations as appropriate and the inventors intend for embodiments of the present disclosure to be practiced otherwise than as specifically described. Accordingly, the scope of the present disclosure includes all modifications and equivalents of the subject matter recited in the claims appended hereto as permitted by applicable law. Moreover, although above-described elements may be described in the context of certain embodiments of the specification, unless stated otherwise or otherwise clear from context, these elements are not mutually exclusive to only those embodiments in which they are described; any combination of the above-described elements in all possible variations thereof is encompassed by the scope of the present disclosure unless otherwise indicated or otherwise clearly contradicted by context.

All references, including publications, patent applications, and patents, cited are hereby incorporated by reference to the same extent as if each reference were individually and specifically indicated to be incorporated by reference and were set forth in its entirety.

1. A self-warming hygienic tissue system, comprising:
a hygienic tissue comprising a first surface and a second surface, wherein:
the first surface comprises a first plurality of ridges and a first plurality of troughs, wherein:
each of the first plurality of ridges are disposed between a pair of troughs of the first plurality of troughs, and
each of the first plurality of troughs are disposed between a pair of ridges of the first plurality of ridges,
the second surface comprises a second plurality of ridges and a second plurality of troughs, wherein:
each of the second plurality of ridges are disposed between a pair of troughs of the second plurality of troughs, and
each of the second plurality of troughs are disposed between a pair of ridges of the second plurality of ridges;
a plurality of interior pockets disposed between the first surface and the second surface, wherein:
each of the plurality of interior pockets comprise an aqueous solution disposed therein,
aqueous solution comprises at least propylene glycol, Aloe barbadensis, citric acid, and glycerin; and
a plurality of microcapsules comprising one or more self-warming solution ingredients, wherein:
the plurality of microcapsules are disposed within the plurality of interior pockets,
the one or more self-warming solution ingredients produce an exothermic chemical reaction when exposed to a threshold temperature,
the threshold temperature is within a range between 85 degrees Fahrenheit and 95 degrees Fahrenheit,
the exothermic chemical reaction of the one or more self-warming solution ingredients causes a surface temperature of the hygienic tissue to rise within a skin temperature range,
the skin temperature range is within a range between 85 degrees Fahrenheit and 100 degrees Fahrenheit, and
the one or more self-warming solution ingredients comprise at least activated charcoal, vermiculite, iron powder, and sodium chloride.

2. The self-warming hygienic tissue system of claim 1, wherein the first surface and the second surface are coupled together at the locations of each of the first plurality of troughs and each of the second plurality of troughs.

3. The self-warming hygienic tissue system of claim 1, wherein each of the first plurality of troughs are coupled to one of the second plurality of troughs.

4. The self-warming hygienic tissue system of claim 1, wherein the first and second plurality of ridges and the first and second plurality of troughs form a shape of a square wave, a sinusoidal wave, a triangular wave, a pulse wave, a sawtooth wave or any combination thereof.

5. The self-warming hygienic tissue system of claim 1, wherein each of the plurality of interior pockets are disposed between a pair of ridges of the first and second plurality of ridges.

6. The self-warming hygienic tissue system of claim 1, wherein the plurality of interior pockets are serially arranged in a first direction.

7. The self-warming hygienic tissue system of claim 6, wherein each of the plurality of interior pockets are arranged parallel one another in a second direction orthogonal to the first direction.

8. The self-warming hygienic tissue system of claim 1, wherein the hygienic tissue is fabricated from cotton, viscose, cellulosic fibers, polyester, polypropylene, polymer resin, wood pulp or any combination thereof.

9. (canceled)

10. (canceled)

11. The self-warming hygienic tissue system of claim 1, wherein each of the plurality of microcapsules comprise a diameter of between 1 micron and 500 microns.

12. The self-warming hygienic tissue system of claim 1, wherein the one or more self-warming solution ingredients contained in each hygienic tissue comprise a total volume of between 3 milliliters and 12 milliliters.

13. The self-warming hygienic tissue system of claim 1, wherein each of the plurality of microcapsules are structurally robust up to a threshold rupturing force.

14. (canceled)

15. (canceled)

16. (canceled)

17. The self-warming hygienic tissue system of claim 1, further comprising a tissue dispensing container housing the hygienic tissue.

18. The self-warming hygienic tissue system of claim 1, wherein the tissue dispensing container comprises a plurality of tissue retention structures.

19. A self-warming hygienic tissue system, comprising:
a hygienic tissue comprising a first surface and a second surface, wherein:
the first surface comprises a first plurality of ridges and a first plurality of troughs forming a first periodic shape,
the second surface comprises a second plurality of ridges and a second plurality of troughs forming a second periodic shape, and
the first and second periodic shapes each form a sinusoidal shape;
a plurality of interior pockets disposed between the first surface and the second surface, wherein:
each of the plurality of interior pockets comprise an aqueous solution disposed therein,
the aqueous solution comprises at least propylene glycol, Aloe barbadensis, citric acid, and glycerin,
the plurality of interior pockets are serially arranged, and
each of the plurality of interior pockets are arranged parallel relative to one another;
a plurality of microcapsules comprising one or more self-warming solution ingredients, wherein:
the one or more self-warming solution ingredients produce an exothermic chemical reaction when exposed to environmental air,
the exothermic chemical reaction of the one or more self-warming solution ingredients causes a surface temperature of the hygienic tissue to rise within a skin temperature range,
the skin temperature range is within a range between 85 degrees Fahrenheit and 100 degrees Fahrenheit, and
the one or more self-warming solution ingredients comprise at least activated charcoal, vermiculite, iron powder, and sodium chloride; and
a tissue dispensing container comprising a closure mechanism having a first retention structure disposed opposite a second retention structure, wherein:
upon dispensation of the hygienic tissue between the first and second retention structures, the hygienic tissue is mechanically abraded by the first and second retention structures, and
the mechanical abrasion of the hygienic tissue causes the one or more self-warming solution ingredients to be exposed to environmental air and undergo the exothermic chemical reaction that raises the surface temperature of the hygienic tissue within the skin temperature range.

20. A self-warming hygienic tissue system, comprising:
a hygienic tissue comprising a first surface and a second surface, wherein:
the first surface comprises a first periodic shape,
the second surface comprises a second periodic shape, and
the first surface is coupled to the second surface;
a plurality of interior pockets disposed between the first periodic shape and the second periodic shape, wherein:
each of the plurality of interior pockets comprise an aqueous solution disposed therein,
the aqueous solution comprises at least propylene glycol, Aloe barbadensis, citric acid, and glycerin,
the plurality of interior pockets are serially arranged,
each of the plurality of interior pockets are arranged parallel relative to one another, and
each of the plurality of interior pockets are disposed between pairs of opposing peaks of the first and second periodic shapes;
a plurality of microcapsules comprising one or more self-warming solution ingredients, wherein:
the plurality of microcapsules are disposed within the plurality of interior pockets,
the one or more self-warming solution ingredients produce an exothermic chemical reaction when exposed to environmental air,
the exothermic chemical reaction raises a surface temperature of the hygienic tissue within a skin temperature range,
the skin temperature range is within a range between 85 degrees Fahrenheit and 100 degrees Fahrenheit, and
the one or more self-warming solution ingredients comprise at least activated charcoal, vermiculite, iron powder, and sodium chloride; and
a tissue dispensing container comprising a closure mechanism having a first retention structure disposed opposite a second retention structure, wherein:
upon dispensation of the hygienic tissue between the first and second retention structures, the hygienic tissue is mechanically abraded by the first and second retention structures, and
the mechanical abrasion of the hygienic tissue causes the one or more self-warming solution ingredients to be exposed to environmental air and undergo the exothermic chemical reaction that raises the surface temperature of the hygienic tissue within the skin temperature range.
